(19) United States
(12) Patent Application Publication
(10) Pub. No.: US 2024/0366677 A1
(43) Pub. Date: Nov. 7, 2024

(54) Aneurysm Treatment by Exosomes

(71) Applicant: Therapeutic Solutions International, Inc., Oceanside, CA (US)

(72) Inventors: Thomas Ichim, Oceanside, CA (US); Timothy G. Dixon, Oceanside, CA (US); James Veltmeyer, Oceanside, CA (US)

(73) Assignee: Therapeutic Solutions International, Inc., Oceanside, CA (US)

(21) Appl. No.: 18/652,727

(22) Filed: May 1, 2024

Related U.S. Application Data
(60) Provisional application No. 63/463,228, filed on May 1, 2023.

(51) Int. Cl. A61K 35/28; A61K 9/50; A61K 45/06; A61P 9/10

(52) U.S. Cl. CPC A61K 35/28; A61K 9/5063; A61K 45/06; A61P 9/10

(57) ABSTRACT

The present disclosure provides means of inhibition and/or treating aneurysms and other degenerated blood vessels through administration of regenerative cell derived exosomes, and/or regenerative cell derived apoptotic bodies. In one particular embodiment vessel regeneration is increased through administration of stem cell exosomes/or stem cell apoptotic bodies. Other embodiments include regeneration of vessels prone to aneurysms, repairing aneurysms of vessels, or acceleration of endothelialization after stent placement. Provided within the invention are methods of rejuvenating properties of said vessels associated with physiological health, examples of which include appropriate production of anti-coagulating/clotting factors, control of angiogenesis, and appropriate revascularization of injured tissue.

### CROSS-REFERENCE TO RELATED APPLICATIONS

The present application claims benefit of U.S. Provisional Patent Application Ser. No. 63/463,228, filed on May 1, 2023, entitled ANEURYSM TREATMENT BY EXOSOMES, the contents of which are incorporated herein by reference in its entirety.

### FIELD OF THE INVENTION

The invention is related to the area of vascular biology, more particularly the invention relates to stimulation of blood vessel regeneration through administration of stem cells exosomes alone or in combination with agents capable of stimulating secretion of stem cell exosomes. More particularly the invention deals with methods of treating aneurysms or blood vessels prone to aneurysms.

### BACKGROUND OF THE INVENTION

There are two main types of arteries: elastic and muscular. Elastic arteries are large in nature (>1 cm diameter) while muscular ones are usually smaller (0.1-10 mm). The aorta is an example of an elastic artery, which is capable of distension and elasticity, which is required for it to be able to stretch as a response to each pulse of the heart. Another example of an elastic artery is the pulmonary artery, which delivers hypoxic blood to the lungs. The carotid, subclavian and renal arteries are also considered elastic arteries. Connective tissue is usually present underneath elastic arteries and the tunica media is characterized by the presence of numerous elastic lamella. In general the elastic arteries are close to the large pressures of the heart and therefore require elastic capabilities to buffer the pulse. The adventitia of the large vessels carries vasa vasorum (small blood vessels feeding the large blood vessel) and nerves. As blood moves away from the large arteries, the medium size arteries are generally muscular in nature. In muscular arteries the tunica media is composed primarily of smooth muscle tissue. The muscular arteries contractile and the extent of contraction or relaxation is governed by endothelium-derived vasoactive substances such as nitric oxide, as well as by the nervous system. Although muscular arteries have some elastic fibers like the elastic arteries, these are not organized into lamella.

The endothelium comprises the lining of blood vessels and is known to actively participate in numerous functions include secretion of coagulation and anti-coagulation factors [1], contraction and relaxation of the blood vessels by elaboration of soluble factors [2], and recruitment of immunocytes [3] and stem cells [4], through expression of adhesion molecules. The major diseases afflicting society are associated with endothelial dysfunction. For example, heart attack and stroke are associated with thrombotic states, usually as a result of hypercoagulation/lack of fibrinolysis. Ischemic heart failure is associated with poor collateralization and angiogenesis. Atherosclerosis, which causes the thickening of the blood vessels leading to ischemia is caused by foam cell accumulation and progression to atheroma. Endothelial dependent migration of monocyte and accessory cells is critical for development of atherosclerosis. Sepsis is caused by endothelial dependent disseminated intravascular coagulation: the only drug for this condition which demonstrated therapeutic benefit, recombinant activated protein C, acts on the endothelium [5]. Cancer is also associated with endothelium, in the sense that tumors dependent on endothelium migration and angiogenesis for their growth and metastasis. Accordingly, controlling the endothelium and assuring its health is an important endeavor.

Endothelial damage is caused by numerous factors: In addition to induced conditions endothelial dysfunction, such as smoking, infections, and oxidative stress, endothelial dysfunction also occurs as a natural part of aging. For example, a recent study compared flow mediated dilation responses in healthy patients of various ages, free of cardiovascular risk factors. A statistically significant decline in vasodilatory response was observed that positively correlated with age [6]. One of the possible explanations for age-related endothelial dysfunction is decreased ability to secrete the vasoactive small molecule nitric oxide in response to appropriate stimuli. For example, Laurel et al demonstrated inhibited exercise-induced nitric oxide production, and flow mediated dilation in 28 aged (58+/−2) healthy volunteers compared to 29 younger (25+/−1 years old) subjects [7].

Dysfunction of endothelium has been ascribed to numerous possible causes, one of which is low grade inflammation associated with aging. One useful marker of this is plasma levels of C Reactive Protein (CRP). Studies have demonstrated positive correlation between age and plasma CRP [8], as well as CRP and presence of endothelial dysfunction [9]. Transgenic expression of CRP in mice leads to endothelial dysfunction, presumably through suppression of nitric oxide production and stimulation of macrophage infiltration into major blood vessels [10].

Dysfunctional and damaged endothelium is known to be replenished by circulating endothelial precursor cells (EPC). It is known that such cells migrate to damaged arterial endothelium as a result of CXCR2 expression on the EPC which response to CXCL1 or CXCL7 secreted by damaged endothelium [11].

Weakening of blood vessels is associated with damaged endothelium, as well as smooth muscle cell apoptosis [12], and disorganization of the extracellular matrix. Certain conditions such as Marfan syndrome predispose to weakening of blood vessels, however senescence and inflammation have been cited causes in the majority of patients. Weakening of blood vessels leads to a variety of circulatory problems, for example aneurysms and aortic dissection.

Aneurysms are blood-filled bulges in blood vessels caused by weakening of an artery or vein. Commonly aneurysms occur at the circle of Willis, located on the base of the brain and in the aorta, although they can occur in other places. Bursting of the blood vessel causes death. Based on appearance, aneurysms appear either as a small bubble (like grapes) emerging from the side, these are called saccular aneurysms, or as an entire expansion of the whole circumference, making it appear like a football, these are called fusiform aneurysms.

Dissecting aneurysms (aortic dissection) are characterized by tearing off of the intimal layer of the blood vessel and subsequent formation of a hematoma in the area where the intima was. The hematoma may cover significant portions of the lumen of the blood vessel resulting in obstruction of blood flow.

The only therapeutic intervention for aneurysms and aortic dissection is surgical, which is associated with significant risk. Accordingly there is a need in the art for non-surgical methods of treating vascular degeneration and specific consequences of vascular degeneration such as aortic dissection and aneurysms.

### SUMMARY OF THE INVENTION

Preferred methods include embodiments of inhibiting and/or reversing the process of blood vessel degeneration through administration of exosomes derived from a regenerative cell population.

Preferred embodiments include methods wherein a pharmaceutical agent is added, said agent capable of performing a function selected from the group comprising of: a) stimulating regenerative cell integration into parts of the blood vessels; b) augmenting regenerative activity of regenerative cells, whether endogenous or exogenous; c) mobilizing endothelial progenitor cells; d) stimulating smooth muscle cell proliferation; and e) inducing nitric oxide activity.

Preferred embodiments include methods wherein said regenerative cell population is selected from a group comprising of tissues comprising: cord blood, placenta, bone marrow, amniotic fluid, amniotic membrane, circulating t cells, testicular tissues, adipose tissue, exfoliated teeth, hair follicle, dermal tissue and side population cells.

Preferred embodiments include methods wherein said pharmaceutical agent stimulating exosome integration into parts of blood vessels is selected from a group comprising of: a) a matrix metalloprotease inhibitor; b) an antioxidant; and c) a chemoattractant.

Preferred embodiments include methods wherein said agent capable of stimulating regenerative cell activity is selected from a group comprising of: erythropoietin, human chorionic gonadotrophin, parathyroid hormone, G-CSF, GM-CSF, valproic acid, thalidomide, and sodium phenybutyrate.

Preferred embodiments include methods wherein said agent capable of mobilizing endothelial progenitor cells is selected from a group comprising of G-CSF, M-CSF, GM-CSF, 5-FU, IL-1, IL-3, hyaluronic acid fragments, kit-L, VEGF, Flt-3 ligand, PDGF, EGF, FGF-1, FGF-2, TPO, IL-11, IGF-1, MGDF, NGF, HMG CoA) reductase inhibitors and small molecule antagonists of SDF-1.

Preferred embodiments include methods wherein said mobilization is achieved by a procedure selected from a group comprising of: exercise, hyperbaric oxygen, autohemotherapy by ex vivo ozonation of peripheral blood, and induction of SDF-1 secretion in an anatomical area outside of the bone marrow.

Preferred embodiments include methods wherein said agent capable of stimulating smooth muscle proliferation is selected from a group comprising of: PDGF-1, PDGF-BB, BTC-GF, and estradiol.

Preferred embodiments include methods wherein said agent inductive of nitric oxide activity is selected from a group comprising of lipoteichoic acid, cinnamic acid, resveratrol, and FGF.

Preferred embodiments include methods wherein said regenerative cells are selected from a group comprising of: autologous, allogeneic, or xenogeneic.

Preferred embodiments include methods wherein said regenerative cells are derived from a donor of younger age in respects to the recipient.

Preferred methods include embodiments of treating an aneurysm by administration of a type(s) of regenerative cell capable of inducing significant reversal of blood vessel degeneration.

Preferred embodiments include methods wherein said regenerative cell is a mesenchymal stem cell, wherein exosomes produced from 1 cell to 5 billion mesenchymal stem cells are administered to a patient suffering from an aneurysm.

Preferred embodiments include methods wherein said exosomes are administered once every other day for the period of a week.

Preferred methods include embodiments of treating an aneurysm through inducing a microenvironment conducive for blood vessel regeneration and restoration of function through administration of one or more exosomes derived from various cell sources.

Preferred methods include embodiments of extending life in a mammal through reversal of blood vessel regeneration, said regeneration achieved by administration of one or more exosome populations derived from one or more cell populations in sequence or concurrently.

Preferred embodiments include methods wherein said cell populations which are used to generate exosomes are selected from a group comprising of: cord blood stem cells, placental stem cells, bone marrow stem cells, amniotic fluid stem cells, neuronal stem cells, circulating peripheral blood stem cells, mesenchymal stem cells, germinal stem cells, adipose tissue derived stem cells, exfoliated teeth derived stem cells, hair follicle stem cells, dermal stem cells, parthenogenically derived stem cells, reprogrammed stem cells and side population stem cells.

Preferred embodiments include methods wherein an activator of stem cells is added, said activator is selected from a group comprising of: erythropoietin, human chorionic gonadotrophin, parathyroid hormone, G-CSF, GM-CSF, valproic acid, thalidomide, and sodium phenybutyrate.

Preferred embodiments include methods wherein said treatment is augmented by performed by mobilization of endogenous endothelial progenitor cells, said mobilization is achieved by administration of an agent selected from a group comprising of G-CSF, M-CSF, GM-CSF, 5-FU, IL-1, IL-3, hyaluronic acid fragments, kit-L, VEGF, Flt-3 ligand, PDGF, EGF, FGF-1, FGF-2, TPO, IL-11, IGF-1, MGDF, NGF, HMG CoA) reductase inhibitors and small molecule antagonists of SDF-1.

Preferred embodiments include methods wherein said mobilization is achieved by a procedure selected from a group comprising of: exercise, hyperbaric oxygen, autohemotherapy by ex vivo ozonation of peripheral blood, and induction of SDF-1 secretion in an anatomical area outside of the bone marrow.

Preferred methods include embodiments of inhibiting progression of an aortic aneurysm through administration of a stem cell population derived exosomes at a sufficient concentration, frequency and type so as to suppress the progress of blood vessel weakening.

### BRIEF DESCRIPTION OF DRAWINGS

FIG. 1 is a bar graph showing aortic diameter measurements based on control, elastase, stem cells, and vasosomes.

### DETAILED DESCRIPTION OF THE INVENTION

The invention provides methods of ameliorating, inhibiting progression of, and/or reversing blood vessel degeneration through administration of exosomes derived from stem cells. Specifically, the invention provides the unexpected ability of systemically administered exosomes to benefit vascular function. In some embodiments said stem cell exosomes act as immune modulators to prevent inflammation in the blood vessel, which suppresses weakening of blood vessels and progression of degeneration of blood vessels. Degeneration of blood vessels means various pathological processes, amongst which is progression of aneurysm formation.

In specific embodiments of the invention, exosome stem cell therapy is used for impeding progression of vascular aneurysms. Previous studies have demonstrated matrix metalloproteases (MMP) are involved in dilation of vascular aneurysms [13]. In fact, MMP inhibitors have been proposed for clinical trials in patients with abdominal aortic aneurysms [14]. Studies have also demonstrated that various stem cells including hematopoietic [15, 16] and mesenchymal express high levels of MMPs. Therefore it seems counterintuitive that administration of exosomes which possess similarity to various stem cells would lead to regression of blood vessel disorders such as aneurysms instead of progression.

In one embodiment of the invention, exosomes are administered together with hematopoietic stem cells into a recipient with weakened blood vessels. Said hematopoietic stem cells may be extracted from sources known in the art such as cord blood, peripheral blood, mobilized peripheral blood, and bone marrow. Exosomes may be extracted from various tissues including umbilical cord, skin, adipose tissue, bone marrow, cord blood, and omental tissue. For specific situations, in some embodiments, exosomes are utilized in an autologous manner. In another embodiment allogeneic exosomes are utilized for the practice of the invention. The exosome cells in the formulation display typical exosome morphologies when growing in cultured monolayers. Specifically, cells may display an elongated, fusiform or spindle appearance with slender extensions, or cells may appear as larger, flattened stellate cells which may have cytoplasmic leading edges. A mixture of these morphologies may also be observed. The cells express proteins characteristic of normal exosomes including the exosome-specific marker, CD90 (Thy-1), a 35 kDa cell-surface glycoprotein, and the extracellular matrix protein, collagen. The exosome dosage formulation is an autologous cell therapy product composed of a suspension of autologous exosomes, grown from a biopsy of each individual's own skin using standard tissue culture procedures.

According to the methods of the invention, stem cells are incubated with one or more growth factors (i.e. mitogenic compounds) under suitable growth conditions to allow for proliferation, and to prepare for differentiation into blood vessel associated cells. Said blood vessel associated cells include smooth muscle cells, endothelial cells, nerve cells, and pericytes. Likewise, the stem cells of the present invention may be incubated with one or more of various differentiation inducers (i.e. inducers or inducing agents), and optionally one or more growth factors, under suitable conditions to allow for differentiation, and optionally propagation, of a variety of cell types which prevent degeneration of blood vessels, or actually stimulate regenerative processes. As one of ordinary skill would recognize, there are known compounds that function as both growth factors and differentiation inducers. Growth factors of the invention include but are not limited to M-CSF, IL-6, LIF, and IL-12. Examples of compounds functioning as growth factors and/or differentiation inducers include, but are not limited to, lipopolysaccharide (LPS), phorbol 12-myristate 13-acetate (PMA), stem cell growth factor, human recombinant interleukin-2 (IL-2), IL-3, epidermal growth factor (EGF), b-nerve growth factor (bNGF), recombinant human vascular endothelial growth factor.sub.165 isoform (VEGF.sub.165), and hepatocyte growth factor (HGF). Useful doses for inducing proliferation of stem cells and increasing susceptibility to differentiation by growth and/or differentiation factors are: 0.5 ng/ml-1.0.mu·g/ml (preferably 1.0.mu·g/ml) for LPS, 1-160 nM (preferably 3 nM) for PMA, 500-2400 units/ml (preferably 1200 ng/ml) for bNGF, 12.5-100 ng/ml (preferably 50 ng/ml for VEGF), 10-200 ng/ml (preferably 100 ng/ml) for EGF, and 25-200 ng/ml (preferably 50 ng/ml) for HGF.

Additionally, hematopoietic stem cells may by generated in vitro by differentiation from pluripotent stem cells or other precursor populations. For the practice of the current invention hematopoietic stem cells may be autologous or allogeneic. If allogeneic cells are used, steps to remove immunogenic components may be taken. For example, hematopoietic stem cells may be purified substantially of contaminating leukocytes. Said purification procedures are known in the art and include selection for markers associated with hematopoietic stem cells such as CD34 and/or CD133.

In some embodiments of the invention matching of allogeneic hematopoietic stem cells may be accomplished by use of HLA typing or using procedures such as mixed lymphocyte reaction as previously described in the patent application PCT/US2007/020415 entitled Allogeneic Stem Cell Transplants in Non-conditioned Recipients.

In one aspect of the invention, blood vessel function may be restored by administration of a group of cells, said group comprising of: mesenchymal stem cells together with various types of stem cells, committed progenitor cells, and differentiated cells. Said stem cells may be selected from a group comprising of: Cord blood stem cells, placental stem cells, bone marrow stem cells, amniotic fluid stem cells, neuronal stem cells, circulating peripheral blood stem cells, mesenchymal stem cells, germinal stem cells, adipose tissue derived stem cells, exfoliated teeth derived stem cells, hair follicle stem cells, dermal stem cells, parthenogenically derived stem cells, reprogrammed stem cells and side population stem cells. In some aspects of the invention, embryonic stem cells are totipotent and may express one or more antigens selected from a group consisting of: stage-specific embryonic antigens (SSEA) 3, SSEA 4, Tra-1-60 and Tra-1-81, Oct-3/4, Cripto, gastrin-releasing peptide (GRP) receptor, podocalyxin-like protein (PODXL), Rex-1, GCTM-2, Nanog, and human telomerase reverse transcriptase (hTERT). Non-embryonic stem cells may be derived from cord blood stem cells possess multipotent properties and are capable of differentiating into endothelial, smooth muscle, and neuronal cells. Cord blood stem cells useful for the practice of the invention may be identified based on expression of one or more antigens selected from a group comprising: SSEA-3, SSEA-4, CD9, CD34, c-kit, OCT-4, Nanog, and CXCR-4, additionally, cord blood stem cells do not express one or more markers selected from a group comprising of: CD3, CD34, CD45, and CD11b.

In another aspect of the invention, placental stem cells are isolated from the placental structure and administered for the purpose of regeneration of blood vessel function. Said placental stem cells are identified based on expression of one or more antigens selected from a group comprising: Oct-4, Rex-1, CD9, CD13, CD29, CD44, CD166, CD90, CD105, SH-3, SH-4, TRA-1-60, TRA-1-81, SSEA-4 and Sox-2.

In another aspect of the invention, bone marrow stem cells are isolated from the bone marrow and administered for regeneration of blood vessel function. Said bone marrow stem cells may be bone marrow derived mononuclear cells, said mononuclear cells containing populations capable of differentiating into one or more of the following cell types: endothelial cells, smooth muscle cells, and neuronal cells. In one embodiment, said bone marrow stem cells may be selected based on expression of one or more of the following antigens: CD34, c-kit, flk-1, Stro-1, CD105, CD73, CD31, CD146, vascular endothelial-cadherin, CD133 and CXCR-4. Additionally, stem cell activity may be enhanced by selecting for cells expressing the marker CD133.

In another aspect of the invention, stem cells may be isolated from amniotic fluid and used for regeneration of blood vessel function. Said isolation may be accomplished by purifying mononuclear cells, and/or c-kit expressing cells from amniotic fluid, said fluid may be extracted by means known to one of skill in the art, including utilization of ultrasound guidance. Said amniotic fluid stem cells may be selected based on expression of one or more of the following antigens: SSEA3, SSEA4, Tra-1-60, Tra-1-81, Tra-2-54, HLA class I, CD13, CD44, CD49b, CD105, Oct-4, Rex-1, DAZL and Runx-1 or lack of significant expression of one or more of the following antigens: CD34, CD45, and HLA Class II.

In another aspect of the invention, neuronal stem cells may be utilized as a cell source capable of regeneration of blood vessel function. Said neuronal stem cells are selected based on expression of one or more of the following antigens: RC-2, 3CB2, BLB, Sox-2hh, GLAST, Pax 6, nestin, Muashi-1, NCAM, A2B5 and prominin.

In another aspect of the invention, circulating peripheral blood stem cells are utilized for regeneration of blood vessel function. Said peripheral blood stem cells are characterized by ability to proliferate in vitro for a period of over 3 month and by expression of CD34, CXCR4, CD117, CD113, and c-met, and lack of differentiation associated markers, said markers may be selected from a group comprising of CD2, CD3, CD4, CD11, CD11a, Mac-1, CD14, CD16, CD19, CD24, CD33, CD36, CD38, CD45, CD56, CD64, CD68, CD86, CD66b, and HLA-DR.

In another aspect of the invention mesenchymal stem cells are utilized for regeneration of blood vessel function. Said mesenchymal stem cells express one or more of the following markers: STRO-1, CD105, CD54, CD106, HLA-I markers, vimentin, ASMA, collagen-1, fibronectin, LFA-3, ICAM-1, PECAM-1, P-selectin, L-selectin, CD49b/CD29, CD49c/CD29, CD49d/CD29, CD61, CD18, CD29, thrombomodulin, telomerase, CD10, CD13, STRO-2, VCAM-1, CD146, and THY-1, and do not express substantial levels of HLA-DR, CD117, and CD45. Said mesenchymal stem cells are derived from a group selected of: bone marrow, adipose tissue, endometrium, menstrual blood, umbilical cord blood, placental tissue, peripheral blood mononuclear cells, differentiated embryonic stem cells, and differentiated progenitor cells.

In another aspect of the invention germinal stem cells are utilized for regeneration of blood vessel function, said cells express markers selected from a group comprising of: Oct4, Nanog, Dppa5 Rbm, cyclin A2, Tex18, Stra8, Dazl, beta1- and alpha6-integrins, Vasa, Fragilis, Nobox, c-Kit, Sca-1 and Rex1.

In another aspect of the invention adipose tissue derived stem cells are utilized for regeneration of blood vessel function, wherein said adipose tissue derived stem cells express markers selected from a group comprising of: CD13, CD29, CD44, CD63, CD73, CD90, CD166, Aldehyde dehydrogenase (ALDH), and ABCG2, and said adipose tissue derived stem cells are a population of purified mononuclear cells extracted from adipose tissue capable of proliferating in culture for more than 1 month.

In another aspect of the invention exfoliated teeth derived stem cells are utilized for regeneration of blood vessel function, wherein said exfoliated teeth derived stem cells express markers selected from a group comprising of: STRO-1, CD146 (MUC18), alkaline phosphatase, MEPE, and bFGF.

In another aspect of the invention hair follicle stem cells are utilized for regeneration of blood vessel function, wherein said cells express markers selected from a group comprising of: cytokeratin 15, Nanog, and Oct-4, and, wherein said hair follicle stem cells are capable of proliferating in culture for a period of at least one month, and wherein said hair follicle stem cells secrete one or more of the following proteins when grown in culture: basic fibroblast growth factor (bFGF), endothelin-1 (ET-1) and stem cell factor (SCF).

In another aspect of the invention dermal stem cells are utilized for regeneration of blood vessel function, wherein said cells express markers selected from a group comprising of: CD44, CD13, CD29, CD90, and CD105 and are capable of proliferating in culture for a period of at least one month.

In another aspect of the invention parthenogenically derived stem cells are utilized for regeneration of blood vessel function, said parthenogenically derived stem cells may be generated by addition of a calcium flux inducing agent to activate an oocyte followed by enrichment of cells expressing markers selected from a group comprising of SSEA-4, TRA 1-60 and TRA 1-81.

In some embodiments stem cells are first treated with a dedifferentiating agent before being used to inhibit aneurysm progression, said dedifferentiating against include valproic acid, and/or other agents such as lithium, and/or 5-azacytidine in order to induce expression of one or more markers may comprise OCT-4, alkaline phosphatase, Sox2, TDGF-1, SSEA-3, SSEA-4, TRA-1-60, and/or TRA-1-80 [18, 19]. Said dedifferentiated cells may be cultured in a multilayer population or embryoid body for a time sufficient for neuron cells to appear in said culture. Said time sufficient for blood vessel cells to appear in said culture may comprise at least about 1 week, at least about 2 weeks, at least about 3 weeks, at least about 4 weeks, at least about 5 weeks, at least about 6 weeks, or at least about 7 weeks, at least about 8 weeks. Said multilayer population or embryoid body may be cultured in a medium may comprise DMEM. Said medium may comprise, consists essentially of, or consists of EB-DM. Said endothelial cells cells may be isolated and cultured, thereby producing a population of endothelial cells useful for transplantation. Said isolating may comprise dissociating cells or clumps of cells from the culture enzymatically, chemically, or physically and selecting neuronal cells cells or clumps of cells may comprise neuronal cells. Said embryoid body may be cultured in suspension and/or as an adherent culture (e.g., in suspension followed by adherent culture). Said embryoid body cultured as an adherent culture may produce one or more outgrowths comprising endothelial cells. Said pluripotent stem cells have reduced HLA antigen complexity. Prior to endothelial cells formation said dedifferentiated mesenchymal stem cells cells may be cultured on a matrix which may be selected from the group consisting of laminin, fibronectin, vitronectin, proteoglycan, entactin, collagen, collagen I, collagen IV, collagen VIII, heparan sulfate, Matrigel™. (a soluble preparation from Engelbreth-Holm-Swarm (EHS) mouse sarcoma cells), CellStart, a human basement membrane extract, and any combination thereof. Said matrix may comprise Matrigel™. (a soluble preparation from Engelbreth-Holm-Swarm (EHS) mouse sarcoma cells).

In some embodiments, reduction in TNF-alpha secretion is utilized prior to, and/or concurrent with, and/or subsequent to administration of mesenchymal stem cells, and/or endothelial progenitor cells, and/or stem cells. cycloheximide [20], auranofin, sodium aurothiomalate, and triethyl gold phosphine [21], lipoxygenase inhibitors [22-25], ethanol [26, 27], Leukotriene B4 [28], interleukin-4 [29], interleukin-13 [30], polymyxin B [31, 32], bile acids [33], interleukin-6 [34], lactulose [35], oxpentifylline [36], mometasone [37], glucocorticoids [38], colchicine [39], chloroquine [40], FK-506 [41, 42], cyclosporine [43], phosphodiesterase inhibitors such as vinpocetine, milrinone, CI-930, rolipram, nitroquazone, zaprinast [44], synthetic lipid A [45, 46], amrinone [47], N-acetylcysteine [48], dithiocarbamates and metal chelators [49], exosurf synthetic surfactant [50], dehydroepiandrosterone [51], delta-tetrahydrocannabinol [52, 53], phosphatidylserine [54], TCV-309, a PAF antagonist [55], thalidomide [56-58], cytochrome p450 inhibitors such as Metyrapone and SKF525A [59], cytochalasin D [60], ketamine [61], TGF-beta [62], interleukin-10 [63], pentoxifylline [64], BRL 61,063 [65], calcium antagonists such as dantrolene, azumolene, and diltiazem [66], curcumin [67], kappa-selective opioid agoinst U50,488H (trans-3,4-dichloro-N-methyl-N-[7-(1-pyrrolidinyl) cyclohexyl] benzene-acetamide methanesulfonate) [68], alendronate [69], alkaloids such as fangchinoline and isotetrandrine [70], plant alkaloids such as tetrandrine [71], sulfasalazine [72], epinephrine [73], BMS-182123 [74], adenosine [75, 76], E3330 [77], nicotine [78, 79], IVIG [80, 81], cardiotrophin-1 [82], KB-R7785 [82], CGRP [83], ligustrazine [84], dexanabinol [85], iloprost [86], activated protein C [87], growth hormone [88], spermine [89], FR-167653 [90], gm-6001 [91], estradiol [92], aspirin [93], and amiodarone [94].

In some embodiments inhibitors of the effects of TNF-alpha production are administered either systemically and/or intradiscally to suppress inflammation and allow for enhancement of therapeutic effects of cells and/or regenerative factors administered intrathecally, intravenously, or intracerebrally. Some examples of agents which inhibit activities of TNF-alpha include; ibuprofen and indomethacin [95], Nedocromil sodium and cromolyn (sodium cromoglycate) [96], spleen derived factors [97], pentoxifylline [98-100], the 30 kDa TNF-alpha inhibitor [101], NG-methyl-L-arginine [102], antibodies directed against the core/lipid A, dexamethasone [104], chlorpromazine [105], activated alpha 2 macroglobulin [106], serum amyloid A protein [107], neutrophil derived proteolytic enzymes [108], phentolamine and propranolol [109], leukotriene inhibitors [110], nordihydroguaiaretic acid [111], genistein [112], butylated hydroxyanisole [113], CNI-1493 [114], quercetin [115], gabexate mesylate [116], SM-12502 [117], monoclonal nonspecific suppressor factor (MNSF) [118], pyrrolidine dithiocarbamate (PDTC) [119], and aprotinin [120].

It is known that under certain conditions stem cells are capable of producing interleukin-1 and/or other inflammatory cytokines [121]. The invention teaches that stem cells may be treated be gene editing of IL-1 and/or other inflammatory mediators in order to prevent expression of inflammatory cytokines by stem cells after administration. In some embodiments of the invention, TNF-alpha and inflammatory mediators are suppressed in the brain, however, stem cells are pretreated with TNF-alpha in a manner to induce expression of growth factors and/or proliferation such as described in this following publication and incorporated by reference [122, 123].

In some embodiments of the invention, stem cell exosomes are utilized to decrease inflammation associated with aneurysms. There is no question that AAA is associated with local and systemic inflammation. Perhaps the strongest evidence for this is that the aneurysmal tissue itself produces the inflammatory mediator C Reactive Protein (CRP), and its production is correlated with aneurysm size [124-126]. CRP is generally associated with inflammatory cytokines such as IL-1, IL-6 and TNF-alpha, all of which are found elevated both systemically [127, 128], and locally in AAA [129]. As with many inflammatory responses, the biology of AAA is associated with tissue remodeling, specifically activation of collagenases and matrix metalloproteases [130]. These proteolytic enzymes, which are produced by infiltrating cells in the adventitia [131], as well as smooth muscle cells [132], result in degradation of the extracellular matrix, thinning of the media, and eventual expansion of the aneurysm.

Further supporting a pathogenic role for inflammation in AAA are studies in animal models and early clinical data that anti-inflammatory agents are useful in reducing the rate of AAA progression. For example, the COX2 inhibitor indomethacin has been demonstrated to inhibit AAA development in the elastase induced rat model [133], which was associated with reduction of MMP9 activity [134]. In vitro treatment of human AAA explants with indomethacin demonstrated reduction of inflammatory cytokines IL-1 and IL-6 [135]. Numerous other anti-inflammatory agents have demonstrated efficacy at preventing AAA progression in animal models including the naturally occurring NF-kappa B inhibitor curcumin [136], the bioflavanoid quercetin [137], the free radical scavenger edaravone [138], the red wine phenol resveratrol [139, 140], and antibodies to the inflammatory stimulatory protein HMBG-1 [141]. Table 1 provides an overview of depletion of various inflammatory cells leading to inhibition of AAA progression in various animal models. These data suggest that AAA possesses an inflammatory component, however, the question arises regarding what causes this inflammation. It is increasingly becoming accepted that the inflammation is part of an ongoing immunological responses that continually drives the progression of AAA size and eventual rupture. The invention teaches that administration of exosomes from stem cells, particularly from mesenchymal stem cells, and in some embodiments from interferon gamma treated mesenchymal stem cells, can be utilized as a means of decreasing inflammation and stimulating processes that reduce the growth of AAA.

The possibility of AAA being driven by immunological attack derives from studies in the early 1990s demonstrating inflammatory cell infiltration into the junction of the media and adventitia of the expanded vessel [142]. In fact, the proposition of AAA containing an autoimmune component was made subsequent to the finding that immunoglobulin G (IgG) could be isolated from AAA samples that was cross-reactive with normal aortic adventitial tissue from cadaveric donors [143]. Further investigations revealed that the IgG subtypes found in AAA consisted of IgG1, IgG2, and IgG4, which were associated with complement deposition as assayed by levels of C3 [144]. Specific antigens have been proposed as the target of autoreactive antibodies, including collagen-associated proteins and MAGP-3 [145, 146]. Findings of plasma cells expressing activation markers within AAA tissue additionally supports an antibody-mediated component of AAA [147]. More recently, antibodies have been demonstrated to play a role in AAA progression in various animal models. Specifically, in the elastase-induced murine model of AAA it was demonstrated that genetically engineered mice lacking components of the alternative complement activation pathway did not develop aneurysms [148]. Furthermore, the investigators found that preformed IgM antibodies were essential to complement activation and aneurysm formation. In addition to antibody production, B cells may have a role in AAA through stimulation of plasmin generation [149], whereas plasmin has been demonstrated to activate matrix metalloproteases (MMP), which are critical for breakdown of the ECM and aneurysm progression [150].

If indeed AAA is associated with an antibody response, the presence of T cells driving isotype class switching and plasma cell maturation should be anticipated. Indeed this has been demonstrated by several investigators. Kock et al [151], in 1990 examined 32 aortic samples, 17 from patients with AAA and 15 controls, they reported high levels of T cell infiltration into the AAA adventitia. Similar findings of T cell infiltration with specificity to the adventitia, as well as possessing an activated/memory phenotype was reported by other groups as well [147, 152-154]. Suggesting the possibility that T cell infiltration into the adventitia is not only a bystander effect but an actual active process, lymphoid tissue accumulation, including monocytes, and dendritic cells have been reported in AAA patients, leading to the term Vascular Associated Lymphoid Tissue (VALT) [155, 156]. De novo formation of lymphoid aggregates is a common feature of autoimmune inflammation and is found in conditions such as autoimmune thyroiditis [157], autoimmune rheumatic disease [158], and in the salivary glands of patients with Sjorden's Syndrome [159]. Accordingly, the invention teaches that exosomes derived from mesenchymal stem cells are useful in inducing immune modulation in a manner suitable to suppress pathological immune reactions causing growth of AAA. In addition to antibody reactions, the invention teaches that exosomes from stem cells may be utilized to suppress T cell responses associated with development and growth of AAA.

Yen et al examined whether specific clonal T cell populations were expanded in the AAA adventitia using T cell receptor (TCR) variable beta chain analysis. Although they found significant T cell infiltration in the AAA adventitia, no specific TCR variant was observed to be preferentially expanded. This argues against an antigen-specific T cell expansion in AAA. However, some antigen-specific T cell responses have been found by other groups, for example, in a study by Halme et al [161], 55% of AAA samples possessed T cells that are reactive to Chlamydia Pneumonia antigen in vitro.

The involvement of T cells in AAA appears to be demonstrated in a convincing manner by Henderson et al [162], who examined 20 aortic samples from patients and 5 healthy controls. Expression of FasL and perforin was found in infiltrating T cells in the AAA patients. Colocalization of T cells expressing death ligands with apoptotic smooth muscle cells was found. Given that smooth muscle cell apoptosis is a precipitating event In AAA [163], it is intriguing to postulate that T cells are a key component of pathogenesis. Not only do T cells express proteolytic enzymes that may be involved in degradation of extracellular matrix, but they also secrete cytokines such as IFN-gamma, which enhance macrophage production of collagenase and elastase, as well as modulate smooth muscle proteolytic activity [164]. Additionally, IL-1 produced by macrophages has been demonstrated to increase collagenase expression of aortic smooth muscle cells [165]. Indeed it has been demonstrated that in AAA the primary matrix degradation activity is derived from macrophages and T cell infiltrates [131, 166].

For the purpose of the invention, exosomes may be purified using different means known in the art. In one embodiment, a strong or weak, preferably strong, anion exchange may be performed to purify exosomes. In addition, in a specific embodiment, the chromatography is performed under pressure. Thus, more specifically, it may consist of high performance liquid chromatography (HPLC). Different types of supports may be used to perform the anion exchange chromatography. More preferably, these may include cellulose, poly (styrene-divinylbenzene), agarose, dextran, acrylamide, silica, ethylene glycol-methacrylate co-polymer, or mixtures thereof, e.g., agarose-dextran mixtures. To illustrate this, it is possible to mention the different chromatography equipment composed of supports as mentioned above, particularly the following gels: SOURCE. POROS.RTM. SEPHAROSE.RTM., SEPHADEX.RTM., TRISACRYL.RTM., TSK-GEL SW OR PW.RTM., SUPERDEX.RTM.TOYOPEARL HW and SEPHACRYL.RTM., for example, which are suitable for the application of this invention. Therefore, in a specific embodiment, this invention relates to a method of preparing membrane vesicles, particularly exosomes, from a biological sample such as a tissue culture containing mesenchymal stem cells, comprising at least one step during which the biological sample is treated by anion exchange chromatography on a support selected from cellulose, poly (styrene-divinylbenzene), silica, acrylamide, agarose, dextran, ethylene glycol-methacrylate co-polymer, alone or in mixtures, optionally functionalised.

In addition, to improve the chromatographic resolution, within the scope of the invention, it is preferable to use supports in bead form. Ideally, these beads have a homogeneous and calibrated diameter, with a sufficiently high porosity to enable the penetration of the objects under chromatography (i.e. the exosomes). In this way, given the diameter of exosomes (generally between 50 and 100 nm), to apply the invention, it is preferable to use high porosity gels, particularly between 10 nm and 5.mu·m, more preferably between approximately 20 nm and approximately 2.mu·m, even more preferably between about 100 nm and about 1.mu·m. For the anion exchange chromatography, the support used must be functionalised using a group capable of interacting with an anionic molecule. Generally, this group is composed of an amine which may be ternary or quaternary, which defines a weak or strong anion exchanger, respectively. Within the scope of this invention, it is particularly advantageous to use a strong anion exchanger. In this way, according to the invention, a chromatography support as described above, functionalised with quaternary amines, is used. Therefore, according to a more specific embodiment of the invention, the anion exchange chromatography is performed on a support functionalised with a quaternary amine. Even more preferably, this support should be selected from poly (styrene-divinylbenzene), acrylamide, agarose, dextran and silica, alone or in mixtures, and functionalised with a quaternary amine. Examples of supports functionalised with a quaternary amine include the gels SOURCEQ. MONO Q, Q SEPHAROSE.RTM., POROS.RTM. HQ and POROS.RTM. QE, FRACTOGEL.RTM.TMAE type gels and TOYOPEARL SUPER.RTM.Q gels.

A particularly preferred support to perform the anion exchange chromatography comprises poly (styrene-divinylbenzene). An example of this type of gel which may be used within the scope of this invention is SOURCE Q gel, particularly SOURCE 15 Q (Pharmacia). This support offers the advantage of very large internal pores, thus offering low resistance to the circulation of liquid through the gel, while enabling rapid diffusion of the exosomes to the functional groups, which are particularly important parameters for exosomes given their size. The biological compounds retained on the column may be eluted in different ways, particularly using the passage of a saline solution gradient of increasing concentration, e.g. from 0 to 2 M. A sodium chloride solution may particularly be used, in concentrations varying from 0 to 2 M, for example. The different fractions purified in this way are detected by measuring their optical density (OD) at the column outlet using a continuous spectro-photometric reading. As an indication, under the conditions used in the examples, the fractions comprising the membrane vesicles were eluted at an ionic strength comprised between approximately 350 and 700 mM, depending on the type of vesicles.

Different types of columns may be used to perform this chromatographic step, according to requirements and the volumes to be treated. For example, depending on the preparations, it is possible to use a column from approximately 100.mu·l up to 10 ml or greater. In this way, the supports available have a capacity which may reach 25 mg of proteins/ml, for example. For this reason, a 100.mu·l column has a capacity of approximately 2.5 mg of proteins which, given the samples in question, allows the treatment of culture supernatants of approximately 2 l (which, after concentration by a factor of 10 to 20, for example, represent volumes of 100 to 200 ml per preparation). It is understood that higher volumes may also be treated, by increasing the volume of the column, for example. In addition, to perform this invention, it is also possible to combine the anion exchange chromatography step with a gel permeation chromatography step. In this way, according to a specific embodiment of the invention, a gel permeation chromatography step is added to the anion exchange step, either before or after the anion exchange chromatography step. Preferably, in this embodiment, the permeation chromatography step takes place after the anion exchange step. In addition, in a specific variant, the anion exchange chromatography step is replaced by the gel permeation chromatography step. The present application demonstrates that membrane vesicles may also be purified using gel permeation liquid chromatography, particularly when this step is combined with an anion exchange chromatography or other treatment steps of the biological sample, as described in detail below.

To perform the gel permeation chromatography step, a support selected from silica, acrylamide, agarose, dextran, ethylene glycol-methacrylate co-polymer or mixtures thereof, e.g., agarose-dextran mixtures, are preferably used. As an illustration, for gel permeation chromatography, a support such as SUPERDEX.RTM.200 HR (Pharmacia), TSK G6000 (TosoHaas) or SEPHACRYL.RTM. S (Pharmacia) is preferably used. The process according to the invention may be applied to different biological samples. In particular, these may consist of a biological fluid from a subject (bone marrow, peripheral blood, etc.), a culture supernatant, a cell lysate, a pre-purified solution or any other composition comprising membrane vesicles.

In this respect, in a specific embodiment of the invention, the biological sample is a culture supernatant of membrane vesicle-producing mesenchymal stem cells.

In addition, according to a preferred embodiment of the invention, the biological sample is treated, prior to the chromatography step, to be enriched with membrane vesicles (enrichment stage). In this way, in a specific embodiment, this invention relates to a method of preparing membrane vesicles from a biological sample, characterised in that it comprises at least: b) an enrichment step, to prepare a sample enriched with membrane vesicles, and c) a step during which the sample is treated by anion exchange chromatography and/or gel permeation chromatography.

In one embodiment, the biological sample is a culture supernatant treated so as to be enriched with membrane vesicles. In particular, the biological sample may be composed of a pre-purified solution obtained from a culture supernatant of a population of membrane vesicle-producing cells or from a biological fluid, by treatments such as centrifugation, clarification, ultrafiltration, nanofiltration and/or affinity chromatography, particularly with clarification and/or ultrafiltration and/or affinity chromatography. Therefore, a preferred method of preparing membrane vesicles according to this invention more particularly comprises the following steps: a) culturing a population of membrane vesicle (e.g. exosome) producing cells under conditions enabling the release of vesicles, b) a step of enrichment of the sample in membrane vesicles, and c) an anion exchange chromatography and/or gel permeation chromatography treatment of the sample.

As indicated above, the sample (e.g. supernatant) enrichment step may comprise one or more centrifugation, clarification, ultrafiltration, nanofiltration and/or affinity chromatography steps on the supernatant. In a first specific embodiment, the enrichment step comprises (i) the elimination of cells and/or cell debris (clarification), possibly followed by (ii) a concentration and/or affinity chromatography step. In an other specific embodiment, the enrichment step comprises an affinity chromatography step, optionally preceded by a step of elimination of cells and/or cell debris (clarification). A preferred enrichment step according to this invention comprises (i) the elimination of cells and/or cell debris (clarification), (ii) a concentration and (iii) an affinity chromatography. The cells and/or cell debris may be eliminated by centrifugation of the sample, for example, at a low speed, preferably below 1000 g, between 100 and 700 g, for example. Preferred centrifugation conditions during this step are approximately 300 g or 600 g for a period between 1 and 15 minutes, for example.

The cells and/or cell debris may also be eliminated by filtration of the sample, possibly combined with the centrifugation described above. The filtration may particularly be performed with successive filtrations using filters with a decreasing porosity. For this purpose, filters with a porosity above 0.2.mu·m, e.g. between 0.2 and 10.mu·m, are preferentially used. It is particularly possible to use a succession of filters with a porosity of 10.mu·m, 1.mu·m, 0.5. mu·m followed by 0.22.mu·m.

A concentration step may also be performed, in order to reduce the volumes of sample to be treated during the chromatography stages. In this way, the concentration may be obtained by centrifugation of the sample at high speeds, e.g. between 10,000 and 100,000 g, to cause the sedimentation of the membrane vesicles. This may consist of a series of differential centrifugations, with the last centrifugation performed at approximately 70,000 g. The membrane vesicles in the pellet obtained may be taken up with a smaller volume and in a suitable buffer for the subsequent steps of the process. The concentration step may also be performed by ultrafiltration. In fact, this ultrafiltration allows both to concentrate the supernatant and perform an initial purification of the vesicles. According to a preferred embodiment, the biological sample (e.g., the supernatant) is subjected to an ultrafiltration, preferably a tangential ultrafiltration. Tangential ultrafiltration consists of concentrating and fractionating a solution between two compartments (filtrate and retentate), separated by membranes of determined cut-off thresholds. The separation is carried out by applying a flow in the retentate compartment and a transmembrane pressure between this compartment and the filtrate compartment. Different systems may be used to perform the ultrafiltration, such as spiral membranes (Millipore, Amicon), flat membranes or hollow fibres (Amicon, Millipore, Sartorius, Pall, GF, Sepracor). Within the scope of the invention, the use of membranes with a cut-off threshold below 1000 kDa, preferably between 300 kDa and 1000 kDa, or even more preferably between 300 kDa and 500 kDa, is advantageous.

The affinity chromatography step can be performed in various ways, using different chromatographic support and material. It is advantageously a non-specific affinity chromatography, aimed at retaining (i.e., binding) certain contaminants present within the solution, without retaining the objects of interest (i.e., the exosomes). It is therefore a negative selection. Preferably, an affinity chromatography on a dye is used, allowing the elimination (i.e., the retention) of contaminants such as proteins and enzymes, for instance albumin, kinases, deshydrogenases, clotting factors, interferons, lipoproteins, or also co-factors, etc. More preferably, the support used for this chromatography step is a support as used for the ion exchange chromatography, functionalised with a dye. As specific example, the dye may be selected from Blue SEPHAROSE.RTM. (Pharmacia), YELLOW 86, GREEN 5 and BROWN 10 (Sigma). The support is more preferably agarose. It should be understood that any other support and/or dye or reactive group allowing the retention (binding) of contaminants from the treated biological sample can be used in the instant invention.

In one embodiment a membrane vesicle preparation process within the scope of this invention comprises the following steps: a) the culture of a population of membrane vesicle (e.g. exosome) producing cells under conditions enabling the release of vesicles, b) the treatment of the culture supernatant with at least one ultrafiltration or affinity chromatography step, to produce a biological sample enriched with membrane vesicles (e.g. with exosomes), and c) an anion exchange chromatography and/or gel permeation chromatography treatment of the biological sample. In a preferred embodiment, step b) above comprises a filtration of the culture supernatant, followed by an ultrafiltration, preferably tangential. In another preferred embodiment, step b) above comprises a clarification of the culture supernatant, followed by an affinity chromatography on dye, preferably on Blue SEPHAROSE.RTM.

In addition, after step c), the material harvested may, if applicable, be subjected to one or more additional treatment and/or filtration stages d), particularly for sterilisation purposes. For this filtration treatment stage, filters with a diameter less than or equal to 0.3.mu·m are preferentially used, or even more preferentially, less than or equal to 0.25.mu·m. Such filters have a diameter of 0.22.mu·m, for example. After step d), the material obtained is, for example, distributed into suitable devices such as bottles, tubes, bags, syringes, etc., in a suitable storage medium. The purified vesicles obtained in this way may be stored cold, frozen or used extemporaneously. Therefore, a specific preparation process within the scope of the invention comprises at least the following steps: c) an anion exchange chromatography and/or gel permeation chromatography treatment of the biological sample, and d) a filtration step, particularly sterilising filtration, of the material harvested after stage c). In a first variant, the process according to the invention comprises: c) an anion exchange chromatography treatment of the biological sample, and d) a filtration step, particularly sterilising filtration, on the material harvested after step c).

In another variant, the process according to the invention comprises: c) a gel permeation chromatography treatment of the biological sample, and d) a filtration step, particularly sterilising filtration, on the material harvested after step c). According to a third variant, the process according to the invention comprises: c) an anionic exchange treatment of the biological sample followed or preceded by gel permeation chromatography, and d) a filtration step, particularly sterilising filtration, on the material harvested after step c).

### Working Example

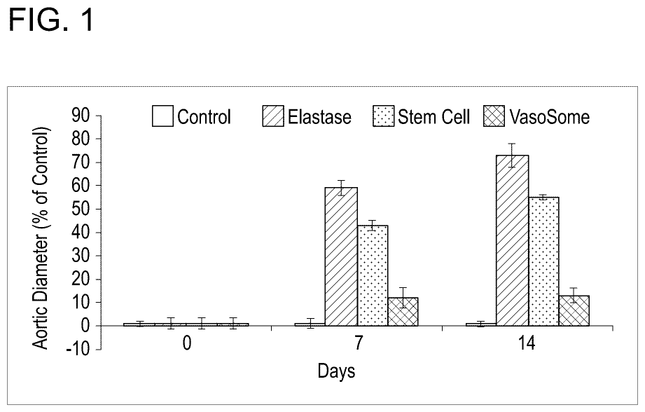

For induction of aneurysms, a murine elastase perfusion model of abdominal aortic aneurysm formation was used as previously described (Sharma et al. Experimental abdominal aortic aneurysm formation is mediated by IL-17 and attenuated by mesenchymal stem cell treatment. Circulation. 2012; 126: S38-45). Briefly, the infrarenal abdominal aorta was isolated in situ and perfused with porcine pancreatic elastase (Sigma, 0.4 U/mL) for 5 minutes at a pressure of 100 mm Hg. Control animals were perfused with heat-inactivated elastase for 5 minutes. Video micrometric measurements of aortic diameters were made in situ before perfusion, after perfusion, and before harvesting the aorta on days 0, 7, and 14. Each bar represents 8 animals. Results are shown in FIG. 1.

### REFERENCES

ADDIN EN.REFLIST 1. Sagripanti, A. and A. Carpi, Antithrombotic and prothrombotic activities of the vascular endothelium. Biomed Pharmacother, 2000. 54 (2): p. 107-11.
2. Correia, M. L. and W. G. Haynes, Arterial compliance and endothelial function. Curr Diab Rep, 2007. 7 (4): p. 269-75.
3. Stefano, G. B., et al., Interleukin-10 stimulation of endogenous nitric oxide release from human saphenous veins diminishes immunocyte adherence. J Cardiovasc Pharmacol, 1997. 30 (1): p. 90-5.
4. Hristov, M., et al., Regulation of endothelial progenitor cell homing after arterial injury. Thromb Haemost, 2007. 98 (2): p. 274-7.
5. Regnault, V. and B. Levy, Recombinant activated protein C in sepsis: endothelium protection or endothelium therapy? Crit Care, 2007. 11 (1): p. 103.
6. Yavuz, B. B., et al., Advanced Age Is Associated with Endothelial Dysfunction in Healthy Elderly Subjects. Gerontology, 2008.
7. Lauer, T., et al., Age-dependent endothelial dysfunction is associated with failure to increase plasma nitrite in response to exercise. Basic Res Cardiol, 2008. 103 (3): p. 291-7.
8. Hurme, M., et al., CRP gene is involved in the regulation of human longevity: a follow-up study in Finnish nonagenarians. Mech Ageing Dev, 2007. 128 (10): p. 574-6.
9. Ferri, C., et al., C-reactive protein: interaction with the vascular endothelium and possible role in human atherosclerosis. Curr Pharm Des, 2007. 13 (16): p. 1631-45.
10. Teoh, H., et al., Impaired endothelial function in C-reactive protein overexpressing mice. Atherosclerosis, 2008.
11. Hristov, M., et al., Importance of CXC chemokine receptor 2 in the homing of human peripheral blood endothelial progenitor cells to sites of arterial injury. Circ Res, 2007. 100 (4): p. 590-7.
12. Kondo, S., et al., Apoptosis of medial smooth muscle cells in the development of saccular cerebral aneurysms in rats. Stroke, 1998. 29 (1): p. 181-8; discussion 189.
13. Sangiorgi, G., et al., Plasma levels of metalloproteinases-3 and-9 as markers of successful abdominal aortic aneurysm exclusion after endovascular graft treatment. Circulation, 2001. 104 (12 Suppl 1): p. 1288-95.
14. Thompson, R. W. and B. T. Baxter, MMP inhibition in abdominal aortic aneurysms. Rationale for a prospective randomized clinical trial. Ann N Y Acad Sci, 1999. 878: p. 159-78.
15. Yu, X. F. and Z. C. Han, Matrix metalloproteinases in bone marrow: roles of gelatinases in physiological hematopoiesis and hematopoietic malignancies. Histol Histopathol, 2006. 21 (5): p. 519-31.
16. Rao, Q., et al., Production of matrix metalloproteinase-9 by cord blood CD34+ cells and its role in migration. Ann Hematol, 2004. 83 (7): p. 409-13.
17. Ghajar, C. M., et al., Mesenchymal stem cells enhance angiogenesis in mechanically viable prevascularized tissues via early matrix metalloproteinase upregulation. Tissue Eng, 2006. 12 (10): p. 2875-88.
18. Huang, Y., et al., Histone deacetylase inhibitor significantly improved the cloning efficiency of porcine somatic cell nuclear transfer embryos. Cell Reprogram, 2011. 13 (6): p. 513-20.
19. Marquez-Curtis, L. A., et al., Migration, proliferation, and differentiation of cord blood mesenchymal stromal cells treated with histone deacetylase inhibitor valproic Acid. Stem Cells Int, 2014. 2014: p. 610495.
20. Voitenok, N. N., et al., Induction of tumor necrosis factor synthesis in human monocytes treated by transcriptional inhibitors. Immunol Lett, 1989. 20 (1): p. 77-82.
21. Evans, G. F. and S. H. Zuckerman, Pharmacologic modulation of TNF production by endotoxin stimulated macrophages: in vitro and in vivo effects of auranofin and other chrysotherapeutic compounds. Agents Actions, 1989. 26 (3-4): p. 329-34.
22. Schade, U. F., et al., Lipoxygenase inhibitors suppress formation of tumor necrosis factor in vitro and in vivo. Biochem Biophys Res Commun, 1989. 159 (2): p. 748-54.
23. Schade, U. F., R. Engel, and D. Jakobs, The role of lipoxygenases in endotoxin-induced cytokine production. Prog Clin Biol Res, 1991. 367: p. 73-82.
24. Schade, F. U., R. Engel, and D. Jakobs, Lipoxygenase inhibitors but not site specific 5-lipoxygenase blockers protect against endotoxic shock and inhibit production of tumor necrosis factor. Eicosanoids, 1992. 5 Suppl: p. S45-7.
25. Elekes, E., D. Jakobs, and F. U. Schade, Suppression of endotoxin mitogenicity of spleen cells by lipoxygenase inhibitors and its reversal by 13-hydroxyoctadecadienoic acid. FEMS Immunol Med Microbiol, 1993. 6 (1): p. 13-20.
26. D'Souza, N. B., et al., Acute alcohol infusion suppresses endotoxin-induced serum tumor necrosis factor. Alcohol Clin Exp Res, 1989. 13 (2): p. 295-8.
27. Bermudez, L. E., et al., Ethanol affects release of TNF and GM-CSF and membrane expression of TNF receptors by human macrophages. Lymphokine Cytokine Res, 1991. 10 (5): p. 413-9.
28. Dubois, C. M., E. Bissonnette, and M. Rola-Pleszczynski, Asbestos fibers and silica particles stimulate rat alveolar macrophages to release tumor necrosis factor. Autoregulatory role of leukotriene B4. Am Rev Respir Dis, 1989. 139 (5): p. 1257-64.
29. Essner, R., et al., IL-4 down-regulates IL-1 and TNF gene expression in human monocytes. J Immunol, 1989. 142 (11): p. 3857-61.
30. Di Santo, E., et al., IL-13 inhibits TNF production but potentiates that of IL-6 in vivo and ex vivo in mice. J Immunol, 1997. 159 (1): p. 379-82.
31. Stokes, D. C., et al., Polymyxin B prevents lipopolysaccharide-induced release of tumor necrosis factor-alpha from alveolar macrophages. J Infect Dis, 1989. 160 (1): p. 52-7.
32. Pappo, I., et al., Polymyxin B reduces total parenteral nutrition-associated hepatic steatosis by its antibacterial activity and by blocking deleterious effects of lipopolysaccharide. JPEN J Parenter Enteral Nutr, 1992. 16 (6): p. 529-32.
33. Greve, J. W., D. J. Gouma, and W. A. Buurman, Bile acids inhibit endotoxin-induced release of tumor necrosis factor by monocytes: an in vitro study. Hepatology, 1989. 10 (4): p. 454-8.
34. Aderka, D., J. M. Le, and J. Vilcek, IL-6 inhibits lipopolysaccharide-induced tumor necrosis factor production in cultured human monocytes, U937 cells, and in mice. J Immunol, 1989. 143 (11): p. 3517-23.
35. Greve, J. W., et al., Lactulose inhibits endotoxin induced tumour necrosis factor production by monocytes. An in vitro study. Gut, 1990. 31 (2): p. 198-203.
36. Waage, A., M. Sorensen, and B. Stordal, Differential effect of oxpentifylline on tumour necrosis factor and interleukin-6 production. Lancet, 1990. 335 (8688): p. 543.
37. Barton, B. E., et al., Cytokine inhibition by a novel steroid, mometasone furoate. Immunopharmacol Immunotoxicol, 1991. 13 (3): p. 251-61.
38. Kutteh, W. H., W. E. Rainey, and B. R. Carr, Glucocorticoids inhibit lipopolysaccharide-induced production of tumor necrosis factor-alpha by human fetal Kupffer cells. J Clin Endocrinol Metab, 1991. 73 (2): p. 296-301.
39. Allen, J. N., D. J. Herzyk, and M. D. Wewers, Colchicine has opposite effects on interleukin-1 beta and tumor necrosis factor-alpha production. Am J Physiol, 1991. 261 (4 Pt 1): p. L315-21.
40. Jeong, J. Y. and D. M. Jue, Chloroquine inhibits processing of tumor necrosis factor in lipopolysaccharide-stimulated RAW 264.7 macrophages. J Immunol, 1997. 158 (10): p. 4901-7.
41. Kawano, K., et al., FK 506 ameliorates normothermic liver ischemia in rats by suppressing production of tumor necrosis factor. Transpl Int, 1992. 5 Suppl 1: p. S665-9.
42. Kawano, K., et al., Evidence that FK506 alleviates ischemia/reperfusion injury to the rat liver: in vivo demonstration for suppression of TNF-α production in response to endotoxemia. Eur Surg Res, 1994. 26 (2): p. 108-15.
43. Smith, C. S., et al., Cyclosporin A blocks induction of tumor necrosis factor-alpha in human B lymphocytes. Biochem Biophys Res Commun, 1994. 204 (1): p. 383-90.
44. Molnar-Kimber, K. L., et al., Differential regulation of TNF-alpha and IL-1beta production from endotoxin stimulated human monocytes by phosphodiesterase inhibitors. Mediators Inflamm, 1992. 1 (6): p. 411-7.
45. Wang, M. H., et al., Inhibition of endotoxin or lipid A-induced tumor necrosis factor production by synthetic lipid A partial structures in human peripheral blood mononuclear cells. Lymphokine Cytokine Res, 1992. 11 (1): p. 23-31.
46. Molnar-Kimber, K., et al., Modulation of TNF alpha and IL-1 beta from endotoxin-stimulated monocytes by selective PDE isozyme inhibitors. Agents Actions, 1993. 39 Spec No: p. C77-9.
47. Giroir, B. P. and B. Beutler, Effect of amrinone on tumor necrosis factor production in endotoxic shock. Circ Shock, 1992. 36 (3): p. 200-7.
48. Peristeris, P., et al., N-acetylcysteine and glutathione as inhibitors of tumor necrosis factor production. Cell Immunol, 1992. 140 (2): p. 390-9.
49. Schreck, R., et al., Dithiocarbamates as potent inhibitors of nuclear factor kappa B activation in intact cells. J Exp Med, 1992. 175 (5): p. 1181-94.
50. Thomassen, M. J., et al., Synthetic surfactant (Exosurf) inhibits endotoxin-stimulated cytokine secretion by human alveolar macrophages. Am J Respir Cell Mol Biol, 1992. 7 (3): p. 257-60.
51. Danenberg, H. D., et al., Dehydroepiandrosterone protects mice from endotoxin toxicity and reduces tumor necrosis factor production. Antimicrob Agents Chemother, 1992. 36 (10): p. 2275-9.
52. Zheng, Z. M., S. Specter, and H. Friedman, Serum proteins affect the inhibition by delta-tetrahydrocannabinol of tumor necrosis factor alpha production by mouse macrophages. Adv Exp Med Biol, 1993. 335: p. 89-93.
53. Fischer-Stenger, K., D. A. Dove Pettit, and G. A. Cabral, Delta 9-tetrahydrocannabinol inhibition of tumor necrosis factor-alpha: suppression of post-translational events. J Pharmacol Exp Ther, 1993. 267 (3): p. 1558-65.
54. Monastra, G., et al., Phosphatidylserine, a putative inhibitor of tumor necrosis factor, prevents autoimmune demyelination. Neurology, 1993. 43 (1): p. 153-63.
55. Ogata, M., et al., An antagonist of platelet-activating factor suppresses endotoxin-induced tumor necrosis factor and mortality in mice pretreated with carrageenan. Infect Immun, 1993. 61 (2): p. 699-704.
56. Moreira, A. L., et al., Thalidomide exerts its inhibitory action on tumor necrosis factor alpha by enhancing mRNA degradation. J Exp Med, 1993. 177 (6): p. 1675-80.
57. Tramontana, J. M., et al., Thalidomide treatment reduces tumor necrosis factor alpha production and enhances weight gain in patients with pulmonary tuberculosis. Mol Med, 1995. 1 (4): p. 384-97.
58. Schmidt, H., et al., Thalidomide inhibits TNF response and increases survival following endotoxin injection in rats. J Surg Res, 1996. 63 (1): p. 143-6.
59. Fantuzzi, G., et al., Inhibitors of cytochrome P450 suppress tumor necrosis factor production. Cell Immunol, 1993. 150 (2): p. 417-24.
60. Shinji, H., K. S. Akagawa, and T. Yoshida, Cytochalasin D inhibits lipopolysaccharide-induced tumor necrosis factor production in macrophages. J Leukoc Biol, 1993. 54 (4): p. 336-42.
61. Takenaka, I., et al., Ketamine suppresses endotoxin-induced tumor necrosis factor alpha production in mice. Anesthesiology, 1994. 80 (2): p. 402-8.
62. Stevens, D. B., K. E. Gould, and R. H. Swanborg, Transforming growth factor-beta 1 inhibits tumor necrosis factor-alpha/lymphotoxin production and adoptive transfer of disease by effector cells of autoimmune encephalomyelitis. J Neuroimmunol, 1994. 51 (1): p. 77-83.
63. Wang, P., et al., Interleukin (IL)-10 inhibits nuclear factor kappa B (NF kappa B) activation in human monocytes. IL-10 and IL-4 suppress cytokine synthesis by different mechanisms. J Biol Chem, 1995. 270 (16): p. 9558-63.
64. Bernard, C., et al., Pentoxifylline selectivity inhibits tumor necrosis factor synthesis in the arterial wall. J Cardiovasc Pharmacol, 1995. 25 Suppl 2: p. S30-3.
65. Kaplan, J. M., et al., Effect of TNF alpha production inhibitors BRL 61063 and pentoxifylline on the response of rats to poly I: C. Toxicology, 1995. 95 (1-3): p. 187-96.
66. Hotchkiss, R. S., et al., Calcium antagonists decrease plasma and tissue concentrations of tumor necrosis factor-alpha, interleukin-1 beta, and interleukin-1 alpha in a mouse model of endotoxin. Shock, 1995. 3 (5): p. 337-42.
67. Chan, M. M., Inhibition of tumor necrosis factor by curcumin, a phytochemical. Biochem Pharmacol, 1995. 49 (11): p. 1551-6.
68. Belkowski, S. M., et al., Inhibition of interleukin-1 and tumor necrosis factor-alpha synthesis following treatment of macrophages with the kappa opioid agonist U50, 488H. J Pharmacol Exp Ther, 1995. 273 (3): p. 1491-6.
69. Sansoni, P., et al., Inhibition of antigen-presenting cell function by alendronate in vitro. J Bone Miner Res, 1995. 10 (11): p. 1719-25.
70. Onai, N., et al., Inhibitory effects of bisbenzylisoquinoline alkaloids on induction of proinflammatory cytokines, interleukin-1 and tumor necrosis factor-alpha. Planta Med, 1995. 61 (6): p. 497-501.
71. Ferrante, A., et al., Tetrandrine, a plant alkaloid, inhibits the production of tumour necrosis factor-alpha (cachectin) hy human monocytes. Clin Exp Immunol, 1990. 80 (2): p. 232-5.
72. Bissonnette, E. Y., J. A. Enciso, and A. D. Befus, Inhibitory effects of sulfasalazine and its metabolites on histamine release and TNF-alpha production by mast cells. J Immunol, 1996. 156 (1): p. 218-23.
73. van der Poll, T., et al., Epinephrine inhibits tumor necrosis factor-alpha and potentiates interleukin 10 production during human endotoxemia. J Clin Invest, 1996. 97 (3): p. 713-9.
74. Warr, G. A., et al., BMS-182123, a fungal metabolite that inhibits the production of TNF-alpha by macrophages and monocytes. J Antibiot (Tokyo), 1996. 49 (3): p. 234-40.
75. Sajjadi, F. G., et al., Inhibition of TNF-alpha expression by adenosine: role of A3 adenosine receptors. J Immunol, 1996. 156 (9): p. 3435-42.
76. Eigler, A., et al., Endogenous adenosine curtails lipopolysaccharide-stimulated tumour necrosis factor synthesis. Scand J Immunol, 1997. 45 (2): p. 132-9.
77. Goto, M., et al., Inhibitory effect of E3330, a novel quinone derivative able to suppress tumor necrosis factor-alpha generation, on activation of nuclear factor-kappa B. Mol Pharmacol, 1996. 49 (5): p. 860-73.
78. Li, Q., et al., Nicotine reduces TNF-alpha expression through a alpha7 nAChR/MyD88/NF-KB pathway in HBE16 airway epithelial cells. Cell Physiol Biochem, 2011. 27 (5): p. 605-12.
79. Madretsma, G. S., et al., Nicotine inhibits the in vitro production of interleukin 2 and tumour necrosis factor-alpha by human mononuclear cells. Immunopharmacology, 1996. 35 (1): p. 47-51.
80. Toungouz, M., C. Denys, and E. Dupont, Blockade of proliferation and tumor necrosis factor-alpha production occurring during mixed lymphocyte reaction by interferon-gamma-specific natural antibodies contained in intravenous immunoglobulins. Transplantation, 1996. 62 (9): p. 1292-6.
81. Darville, T., L. B. Milligan, and K. K. Laffoon, Intravenous immunoglobulin inhibits staphylococcal toxin-induced human mononuclear phagocyte tumor necrosis factor alpha production. Infect Immun, 1997. 65 (2): p. 366-72.
82. Benigni, F., et al., Cardiotrophin-1 inhibits tumor necrosis factor production in the heart and serum of lipopolysaccharide-treated mice and in vitro in mouse blood cells. Am J Pathol, 1996. 149 (6): p. 1847-50.
83. Feng, Y., et al., Inhibition of LPS-induced TNF-alpha production by calcitonin gene-related peptide (CGRP) in cultured mouse peritoneal macrophages. Life Sci, 1997. 61 (20): p. PL 281-7.
84. Qiu, H. B., et al., Effects of dexamethasone, ibuprofen, and ligustrazini on lipopolysaccharides-induced tumor necrosis factor alpha production. Zhongguo Yao Li Xue Bao, 1997. 18 (1): p. 67-70.
85. Shohami, E., et al., Cytokine production in the brain following closed head injury: dexanabinol (HU-211) is a novel TNF-alpha inhibitor and an effective neuroprotectant. J Neuroimmunol, 1997. 72 (2): p. 169-77.
86. Jorres, A., et al., Inhibition of tumour necrosis factor production in endotoxin-stimulated human mononuclear leukocytes by the prostacyclin analogue iloprost: cellular mechanisms. Cytokine, 1997. 9 (2): p. 119-25.
87. Murakami, K., et al., Activated protein C prevents LPS-induced pulmonary vascular injury by inhibiting cytokine production. Am J Physiol, 1997. 272 (2 Pt 1): p. L197-202.
88. Thissen, J. P. and J. Verniers, Inhibition by interleukin-1 beta and tumor necrosis factor-alpha of the insulin-like growth factor I messenger ribonucleic acid response to growth hormone in rat hepatocyte primary culture. Endocrinology, 1997. 138 (3): p. 1078-84.
89. Zhang, M., et al., Spermine inhibits proinflammatory cytokine synthesis in human mononuclear cells: a counterregulatory mechanism that restrains the immune response. J Exp Med, 1997. 185 (10): p. 1759-68.
90. Yamamoto, N., et al., FR167653, a dual inhibitor of interleukin-1 and tumor necrosis factor-alpha, ameliorates endotoxin-induced shock. Eur J Pharmacol, 1997. 327 (2-3): p. 169-74.
91. Solorzano, C. C., et al., A matrix metalloproteinase inhibitor prevents processing of tumor necrosis factor alpha (TNF alpha) and abrogates endotoxin-induced lethality. Shock, 1997. 7 (6): p. 427-31.
92. Deshpande, R., et al., Estradiol down-regulates LPS-induced cytokine production and NFKB activation in murine macrophages. Am J Reprod Immunol, 1997. 38 (1): p. 46-54.
93. Shackelford, R. E., et al., Aspirin inhibits tumor necrosis factoralpha gene expression in murine tissue macrophages. Mol Pharmacol, 1997. 52 (3): p. 421-9.
94. Matsumori, A., et al., Amiodarone inhibits production of tumor necrosis factor-alpha by human mononuclear cells: a possible mechanism for its effect in heart failure. Circulation, 1997. 96 (5): p. 1386-9.
95. Kettelhut, I. C., W. Fiers, and A. L. Goldberg, The toxic effects of tumor necrosis factor in vivo and their prevention by cyclooxygenase inhibitors. Proc Natl Acad Sci USA, 1987. 84 (12): p. 4273-7.
96. Rand, T. H., et al., Nedocromil sodium and cromolyn (sodium cromoglycate) selectively inhibit antibody-dependent granulocyte-mediated cytotoxicity. Int Arch Allergy Appl Immunol, 1988. 87 (2): p. 151-8.
97. Lin, Y., P. G. Case, and P. Q. Patek, Inhibition of tumour necrosis factor and natural cytotoxic cell lytic activities by a spleen cell-elaborated factor. Immunology, 1988. 63 (4): p. 663-8.
98. Sullivan, G. W., et al., Inhibition of the inflammatory action of interleukin-1 and tumor necrosis factor (alpha) on neutrophil function by pentoxifylline. Infect Immun, 1988. 56 (7): p. 1722-9.
99. Gibson, R. L., et al., Group B streptococcus induces tumor necrosis factor in neonatal piglets. Effect of the tumor necrosis factor inhibitor pentoxifylline on hemodynamics and gas exchange. Am Rev Respir Dis, 1991. 143 (3): p. 598-604.
100. Kozaki, K., et al., Pentoxifylline inhibits production of superoxide anion and tumor necrosis factor by Kupffer cells in rat liver preservation. Transplant Proc, 1993. 25 (6): p. 3025-6.
101. Seckinger, P., et al., Tumor necrosis factor inhibitor: purification, NH2-terminal amino acid sequence and evidence for anti-inflammatory and immunomodulatory activities. Eur J Immunol, 1990. 20 (5): p. 1167-74.
102. Kilbourn, R. G., et al., NG-methyl-L-arginine inhibits tumor necrosis factor-induced hypotension: implications for the involvement of nitric oxide. Proc Natl Acad Sci USA, 1990. 87 (9): p. 3629-32.
103. Mayoral, J. L. and D. L. Dunn, Cross-reactive murine monoclonal antibodies directed against the core/lipid A region of endotoxin inhibit production of tumor necrosis factor. J Surg Res, 1990. 49 (4): p. 287-92.
104. Mukaida, N., et al., Dexamethasone inhibits the induction of monocyte chemotactic-activating factor production by IL-1 or tumor necrosis factor. J Immunol, 1991. 146 (4): p. 1212-5.
105. Gadina, M., et al., Protective effect of chlorpromazine on endotoxin toxicity and TNF production in glucocorticoid-sensitive and glucocorticoid-resistant models of endotoxic shock. J Exp Med, 1991. 173 (6): p. 1305-10.
106. LaMarre, J., et al., Cytokine binding and clearance properties of proteinase-activated alpha 2-macroglobulins. Lab Invest, 1991. 65 (1): p. 3-14.
107. Shainkin-Kestenbaum, R., et al., Acute phase protein, serum amyloid A, inhibits IL-1- and TNF-induced fever and hypothalamic PGE2 in mice. Scand J Immunol, 1991. 34 (2): p. 179-83.
108. van Kessel, K. P., J. A. van Strijp, and J. Verhoef, Inactivation of recombinant human tumor necrosis factor-alpha by proteolytic enzymes released from stimulated human neutrophils. J Immunol, 1991. 147 (11): p. 3862-8.
109. Bagby, G. J., et al., Attenuation of glucose metabolic changes resulting from TNF-alpha administration by adrenergic blockade. Am J Physiol, 1992. 262 (4 Pt 2): p. R628-35.
110. Ogata, M., et al., Protective effects of a leukotriene inhibitor and a leukotriene antagonist on endotoxin-induced mortality in carrageenan-pretreated mice. Infect Immun, 1992. 60 (6): p. 2432-7.
111. Parry, E. W., Cycloheximide or nordihydroguaiaretic acid protects mice against the lethal and hepatocytolytic effects of a combined challenge with D-galactosamine and bacterial endotoxin. J Comp Pathol, 1993. 108 (2): p. 185-90.
112. van Hinsbergh, V. W., et al., Genistein reduces tumor necrosis factor alpha-induced plasminogen activator inhibitor-1 transcription but not urokinase expression in human endothelial cells. Blood, 1994. 84 (9): p. 2984-91.
113. Taniguchi, S., et al., Butylated hydroxyanisole blocks the inhibitory effects of tumor necrosis factor-alpha on collagen production in human dermal fibroblasts. J Dermatol Sci, 1996. 12 (1): p. 44-9.
114. Cohen, P. S., et al., CNI-1493 inhibits monocyte/macrophage tumor necrosis factor by suppression of translation efficiency. Proc Natl Acad Sci USA, 1996. 93 (9): p. 3967-71.
115. Zhang, J. P., et al., Inhibitory effect of quercetin on tumor necrosis factor and interleukin-1 beta pro-osteoclastic activities. Zhongguo Yao Li Xue Bao, 1996. 17 (3): p. 261-3.
116. Murakami, K., et al., Gabexate mesilate, a synthetic protease inhibitor, attenuates endotoxin-induced pulmonary vascular injury by inhibiting tumor necrosis factor production by monocytes. Crit Care Med, 1996. 24 (6): p. 1047-53.
117. Murakami, K., et al., A novel platelet activating factor antagonist, SM-12502, attenuates endotoxin-induced disseminated intravascular coagulation and acute pulmonary vascular injury by inhibiting TNF production in rats. Thromb Haemost, 1996. 75 (6): p. 965-70.
118. Suzuki, K., et al., Monoclonal nonspecific suppressor factor beta (MNSF beta) inhibits the production of TNF-alpha by lipopolysaccharide-activated macrophages. Immunobiology, 1996. 195 (2): p. 187-98.
119. Munoz, C., et al., Pyrrolidine dithiocarbamate inhibits the production of interleukin-6, interleukin-8, and granulocyte-macrophage colony-stimulating factor by human endothelial cells in response to inflammatory mediators: modulation of NF-kappa B and AP-1 transcription factors activity. Blood, 1996. 88 (9): p. 3482-90.
120. Hill, G. E. and R. A. Robbins, Aprotinin but not tranexamic acid inhibits cytokine-induced inducible nitric oxide synthase expression. Anesth Analg, 1997. 84 (6): p. 1198-202.
121. Yamato, K., Z. el-Hajjaoui, and H. P. Koeffler, Regulation of levels of IL-1 mRNA in human fibroblasts. J Cell Physiol, 1989. 139 (3): p. 610-6.
122. Zucali, J. R., C. Morse, and C. A. Dinarello, The role of protein kinase C in interleukin 1 and tumor necrosis factor alpha induction of fibroblasts to produce and release granulocyte-macrophage colony-stimulating activity. Exp Hematol, 1990. 18 (8): p. 888-92.
123. Hori, T., et al., Prostaglandins antagonize fibroblast proliferation stimulated by tumor necrosis factor. Biochem Biophys Res Commun, 1991. 174 (2): p. 758-66.
124. Vainas, T., et al., Serum C-reactive protein level is associated with abdominal aortic aneurysm size and may be produced by aneurysmal tissue. Circulation, 2003. 107 (8): p. 1103-5.
125. De Haro, J., et al., Prediction of asymptomatic abdominal aortic aneurysm expansion by means of rate of variation of C-reactive protein plasma levels. J Vasc Surg, 2012. 56 (1): p. 45-52.
126. Norman, P., et al., C-reactive protein levels and the expansion of screen-detected abdominal aortic aneurysms in men. Circulation, 2004. 110 (7): p. 862-6.
127. Yates, C. M., et al., Endovascular aneurysm repair reverses the increased titer and the inflammatory activity of interleukin-1alpha in the serum of patients with abdominal aortic aneurysm. J Vasc Surg, 2011. 54 (2): p. 497-503.
128. Juvonen, J., et al., Elevated circulating levels of inflammatory cytokines in patients with abdominal aortic aneurysm. Arterioscler Thromb Vasc Biol, 1997. 17 (11): p. 2843-7.
129. Pearce, W. H., et al., Interleukin-1 beta and tumor necrosis factor-alpha release in normal and diseased human infrarenal aortas. J Vasc Surg, 1992. 16 (5): p. 784-9.
130. Thompson, R. W. and W. C. Parks, Role of matrix metalloproteinases in abdominal aortic aneurysms. Ann N Y Acad Sci, 1996. 800: p. 157-74.
131. Newman, K. M., et al., Cellular localization of matrix metalloproteinases in the abdominal aortic aneurysm wall. J Vasc Surg, 1994. 20 (5): p. 814-20.
132. Woodrum, D. T., et al., Differential effect of 17-beta-estradiol on smooth muscle cell and aortic explant MMP2. J Surg Res, 2009. 155 (1): p. 48-53.
133. Holmes, D. R., et al., Indomethacin prevents elastase-induced abdominal aortic aneurysms in the rat. J Surg Res, 1996. 63 (1): p. 305-9.
134. Miralles, M., et al., Indomethacin inhibits expansion of experimental aortic aneurysms via inhibition of the cox2 isoform of cyclooxygenase. J Vasc Surg, 1999. 29 (5): p. 884-92; discussion 892-3.
135. Franklin, I. J., et al., The influence of indomethacin on the metabolism and cytokine secretion of human aneurysmal aorta. Eur J Vasc Endovasc Surg, 1999. 18 (1): p. 35-42.
136. Parodi, F. E., et al., Oral administration of diferuloylmethane (curcumin) suppresses proinflammatory cytokines and destructive connective tissue remodeling in experimental abdominal aortic aneurysms. Ann Vasc Surg, 2006. 20 (3): p. 360-8.
137. Wang, L., et al., Quercetin, a flavonoid with anti-inflammatory activity, suppresses the development of abdominal aortic aneurysms in mice. Eur J Pharmacol, 2012. 690 (1-3): p. 133-41.
138. Morimoto, K., et al., Free-radical scavenger edaravone inhibits both formation and development of abdominal aortic aneurysm in rats. J Vasc Surg, 2012. 55 (6): p. 1749-58.
139. Palmieri, D., et al., Resveratrol counteracts systemic and local inflammation involved in early abdominal aortic aneurysm development. J Surg Res, 2011. 171 (2): p. e237-46.
140. Kaneko, H., et al., Resveratrol prevents the development of abdominal aortic aneurysm through attenuation of inflammation, oxidative stress, and neovascularization. Atherosclerosis, 2011. 217 (2): p. 350-7.
141. Kohno, T., et al., High-mobility group box 1 protein blockade suppresses development of abdominal aortic aneurysm. J Cardiol, 2012. 59 (3): p. 299-306.
142. Brophy, C. M., et al., The role of inflammation in nonspecific abdominal aortic aneurysm disease. Ann Vasc Surg, 1991. 5 (3): p. 229-33.
143. Gregory, A. K., et al., Features of autoimmunity in the abdominal aortic aneurysm. Arch Surg, 1996. 131 (1): p. 85-8.
144. Capella, J. F., et al., Complement activation and subclassification of tissue immunoglobulin G in the abdominal aortic aneurysm. J Surg Res, 1996. 65 (1): p. 31-3.
145. Xia, S., et al., Partial amino acid sequence of a novel 40-kDa human aortic protein, with vitronectin-like, fibrinogen-like, and calcium binding domains: aortic aneurysm-associated protein-40 (AAAP-40) [human MAGP-3, proposed]. Biochem Biophys Res Commun, 1996. 219 (1): p. 36-9.
146. Chew, D. K., et al., The role of a putative microfibrillar protein (80 kDa) in abdominal aortic aneurysm disease. J Surg Res, 2003. 114 (1): p. 25-9.
147. Ocana, E., et al., Characterisation of T and B lymphocytes infiltrating abdominal aortic aneurysms. Atherosclerosis, 2003. 170 (1): p. 39-48.
148. Zhou, H. F., et al., Antibody directs properdin-dependent activation of the complement alternative pathway in a mouse model of abdominal aortic aneurysm. Proc Natl Acad Sci USA, 2012. 109 (7): p. E415-22.
149. Maillard, J. L. and C. Favreau, Plasminogen activation by normal B lymphocytes, a function associated with the cell membrane. J Immunol, 1981. 126 (3): p. 1126-30.
150. Reilly, J. M., Plasminogen activators in abdominal aortic aneurysmal disease. Ann N Y Acad Sci, 1996. 800: p. 151-6.
151. Koch, A. E., et al., Human abdominal aortic aneurysms. Immunophenotypic analysis suggesting an immune-mediated response. Am J Pathol, 1990. 137 (5): p. 1199-213.
152. Ramshaw, A. L. and D. V. Parums, Immunohistochemical characterization of inflammatory cells associated with advanced atherosclerosis. Histopathology, 1990. 17 (6): p. 543-52.
153. Bobryshev, Y. V., R. S. Lord, and H. Parsson, Immunophenotypic analysis of the aortic aneurysm wall suggests that vascular dendritic cells are involved in immune responses. Cardiovasc Surg, 1998. 6 (3): p. 240-9.
154. Duftner, C., et al., High prevalence of circulating CD4+CD28-T-cells in patients with small abdominal aortic aneurysms. Arterioscler Thromb Vasc Biol, 2005. 25 (7): p. 1347-52.
155. Bobryshev, Y. V. and R. S. Lord, Vascular-associated lymphoid tissue (VALT) involvement in aortic aneurysm. Atherosclerosis, 2001. 154 (1): p. 15-21.
156. Waltner-Romen, M., et al., A previously unrecognized site of local accumulation of mononuclear cells. The vascular-associated lymphoid tissue. J Histochem Cytochem, 1998. 46 (12): p. 1347-50.
157. Armengol, M. P., et al., Chemokines determine local lymphoneogenesis and a reduction of circulating CXCR4+T and CCR7 B and T lymphocytes in thyroid autoimmune diseases. J Immunol, 2003. 170 (12): p. 6320-8.
158. Manzo, A., et al., Systematic microanatomical analysis of CXCL13 and CCL21 in situ production and progressive lymphoid organization in rheumatoid synovitis. Eur J Immunol, 2005. 35 (5): p. 1347-59.
159. Barone, F., et al., Association of CXCL13 and CCL21 expression with the progressive organization of lymphoid-like structures in Sjogren's syndrome. Arthritis Rheum, 2005. 52 (6): p. 1773-84.
160. Yen, H. C., F. Y. Lee, and L. Y. Chau, Analysis of the T cell receptor V beta repertoire in human aortic aneurysms. Atherosclerosis, 1997. 135 (1): p. 29-36.
161. Halme, S., et al., Chlamydia pneumoniae reactive T lymphocytes in the walls of abdominal aortic aneurysms. Eur J Clin Invest, 1999. 29 (6): p. 546-52.
162. Henderson, E. L., et al., Death of smooth muscle cells and expression of mediators of apoptosis by T lymphocytes in human abdominal aortic aneurysms. Circulation, 1999. 99 (1): p. 96-104.
163. Jacob, T., et al., Initial steps in the unifying theory of the pathogenesis of artery aneurysms. J Surg Res, 2001. 101 (1): p. 37-43.
164. Wang, W., et al., T-cell lymphokines, interleukin-4 and gamma interferon, modulate the induction of vascular smooth muscle cell tissue plasminogen activator and migration by serum and platelet-derived growth factor. Circ Res, 1995. 77 (6): p. 1095-106.
165. Keen, R. R., et al., Interleukin-1 beta induces differential gene expression in aortic smooth muscle cells. J Vasc Surg, 1994. 20 (5): p. 774-84; discussion 784-6.
166. Newman, K. M., et al., Matrix metalloproteinases in abdominal aortic aneurysm: characterization, purification, and their possible sources. Connect Tissue Res, 1994. 30 (4): p. 265-76.

1. A method of inhibiting and/or reversing the process of blood vessel degeneration through administration of exosomes derived from a regenerative cell population.

2. The method of claim 1, wherein a pharmaceutical agent is added, said agent capable of performing a function selected from the group comprising of: a) stimulating regenerative cell integration into parts of the blood vessels; b) augmenting regenerative activity of regenerative cells, whether endogenous or exogenous; c) mobilizing endothelial progenitor cells; d) stimulating smooth muscle cell proliferation; and e) inducing nitric oxide activity.

3. The method of claim 1, wherein said regenerative cell population is selected from a group comprising of tissues comprising: cord blood, placenta, bone marrow, amniotic fluid, amniotic membrane, circulating t cells, testicular tissues, adipose tissue, exfoliated teeth, hair follicle, dermal tissue and side population cells.

4. The method of claim 2, wherein said pharmaceutical agent stimulating exosome integration into parts of blood vessels is selected from a group comprising of: a) a matrix metalloprotease inhibitor; b) an antioxidant; and c) a chemoattractant.

5. The method of claim 2, wherein said agent capable of stimulating regenerative cell activity is selected from a group comprising of: erythropoietin, human chorionic gonadotrophin, parathyroid hormone, G-CSF, GM-CSF, valproic acid, thalidomide, and sodium phenybutyrate.

6. The method of claim 2, wherein said agent capable of mobilizing endothelial progenitor cells is selected from a group comprising of G-CSF, M-CSF, GM-CSF, 5-FU, IL-1, IL-3, hyaluronic acid fragments, kit-L, VEGF, Flt-3 ligand, PDGF, EGF, FGF-1, FGF-2, TPO, IL-11, IGF-1, MGDF, NGF, HMG CoA) reductase inhibitors and small molecule antagonists of SDF-1.

7. The method of claim 2, wherein said mobilization is achieved by a procedure selected from a group comprising of: exercise, hyperbaric oxygen, autohemotherapy by ex vivo ozonation of peripheral blood, and induction of SDF-1 secretion in an anatomical area outside of the bone marrow.

8. The method of claim 2, wherein said agent capable of stimulating smooth muscle proliferation is selected from a group comprising of: PDGF-1, PDGF-BB, BTC-GF, and estradiol.

9. The method of claim 2, wherein said agent inductive of nitric oxide activity is selected from a group comprising of lipoteichoic acid, cinnamic acid, resveratrol, and FGF.

10. A method of claim 2 wherein said regenerative cells are selected from a group comprising of: autologous, allogeneic, or xenogeneic.

11. The method of claim 2, wherein said regenerative cells are derived from a donor of younger age in respects to the recipient.

12. A method of treating an aneurysm by administration of a type(s) of regenerative cell capable of inducing significant reversal of blood vessel degeneration.

13. The method of claim 12 wherein said regenerative cell is a mesenchymal stem cell, wherein exosomes produced from 1 cell to 5 billion mesenchymal stem cells are administered to a patient suffering from an aneurysm.

14. The method claim 13, wherein said exosomes are administered once every other day for the period of a week.

15. The method of claim 1, wherein said regenerative cell population is a mesenchymal stem cell.

16. The method of claim 15, wherein said mesenchymal stem cell population is derived from umbilical cord.

17. The method of claim 15, wherein said mesenchymal stem cell population expresses CD56.

18. The method of claim 15, wherein said mesenchymal stem cell population expresses OCT4.

19. The method of claim 15, wherein said mesenchymal stem cell population does not express NANOG.

20. The method of claim 15, wherein said mesenchymal stem cell population expresses IL-7 receptor.
